(19) United States
(12) Patent Application Publication
(10) Pub. No.: US 2025/0122313 A1
(43) Pub. Date: Apr. 17, 2025

(54) METHODS FOR PRODUCING GLYCATED CHITOSANS

(71) Applicant: Immunophotonics, Inc., St. Louis, MO (US)

(72) Inventors: Joseph Raker, St. Louis, MO (US); Tomas Hode, St. Louis, MO (US); Abigail Delawder, St. Louis, MO (US); Luciano Alleruzzo, St. Louis, MO (US)

(21) Appl. No.: 18/693,262

(22) PCT Filed: Sep. 21, 2022
(86) PCT No.: PCT/US2022/044229

§ 371 Date: Mar. 19, 2024

(51) Int. Cl. C08B 37/08

(52) U.S. Cl. CPC C08B 37/003

(57) ABSTRACT

The present disclosure provides a process for preparing glycated chitosan and compositions comprising glycated chitosan.

### RELATED APPLICATIONS

This application claims the benefit of priority to U.S. Provisional Patent Application No. 63/246,417, filed on Sep. 21, 2021, the contents of which are fully incorporated by reference herein.

### BACKGROUND

Immunotherapy has been used with some success for systemic treatment of chemotherapy-resistant cancers. Cancer immunotherapy is based on inducing or boosting an immune response against the tumor(s). Specific methodologies to accomplish this have so far included cancer vaccines, modulation of immunity by cytokines or antibodies, adoptive transfer of immune cells, and others.

One such methodology to induce a robust and prolonged anti-tumor response (and hence effective overall clinical outcome) is to combine tumor-specific antigens with adjunct agents that either prolong antigen delivery (“depot effect”) and/or induce an appropriate immune stimulating effect, and thus enhance an immunological response against the cancer. One such adjunct agent is the semisynthetic cationic carbohydrate biopolymer glycated chitosan (GC). Key advantageous properties of GC include improved water solubility and immune stimulation over base chitosans, sterile filterability, biocompatibility, and capability to serve as physiologically compatible carrier of other therapeutic agents (e.g. nanomaterials).

GC is prepared by attaching saccharide molecules to chitosan. Chitosan itself is produced from chitin, a structural component in many organisms, found for example in exoskeletons in arthropods, such as crustaceans and insects, and as cell walls in fungi. The biopolymer chitin is a linear homopolymer composed of N-acetylglucosamine units joined by β 1→4 glycosidic bonds. Chitosan, which is partially deacetylated chitin, is the most studied member of this class of biopolymer-derived compounds. The presence of primary amino groups in chitosan facilitates a number of approaches for chemical modifications designed to achieve their solubilization and to impart special properties for specific applications.

One such chemical modification is realized via the synthesis of GC and the manufacturing of GCs, in which chitosan and a reducing sugar are the starting materials used to manufacture the GC compounds via a reductive amination reaction involving the free amino groups of chitosan and the carbonyl groups of the reducing monosaccharides and/or oligosaccharides as described, for example, in Sashiwa et al., Prog. Polym. Sci. 29 (2004), 887-908, which is fully incorporated by reference herein.

Historically, and generally speaking, the manufacture of glycated chitosan involved a reducing monosaccharide (e.g. glucose, galactose, ribose, etc.), or an equivalent amount of a reducing monosaccharide and/or oligosaccharide that was dissolved in a dilute aqueous acidic solution, and mixed with chitosan, which allowed the formation of an imine between the sugar and the amino groups of chitosan. Thereafter, the homogeneous mixture was reduced using an aqueous mixture of sodium borohydride in sodium hydroxide under stirring.

After this stirring step, the solution was acidified to a pH of 5.5 by the dropwise addition of acetic acid under further stirring to decompose excess borohydride. As described in U.S. Pat. No. 5,747,475 (“Chitosan-Derived Biomaterials”), particulate matter formed during the reaction, together with foam and a gel. To break up the foam and separate the particulates from the glycated chitosan gel, the solution was centrifuged, the supernatant was decanted off, and the solids re-suspended and centrifuged again. This final step was repeated several times, followed by dialysis to purify the GC.

Although some work has been done to improve overall yield during the laboratory-scale manufacture of GC, for example as described in U.S. Pat. No. 5,747,475, conventional preparation methods continue to be plagued by gelation and particulate matter formation that are difficult to remove using conventional purification methods. GCs are therefore extremely difficult to manufacture and purify on a larger scale. Moreover, GCs prepared by conventional methods, as described in U.S. Pat. No. 5,747,475, are nearly impossible to sterile filter because of gelling and particulate formation, rendering them infeasible for industrial manufacturing according to Current Good Manufacturing Practices (cGMP) and therefore unsuitable for human use.

To date, consistent manufacture with little or no batch-to-batch variability remains a challenge. Thus, there is a significant unmet need for a better process to manufacture glycated chitosan without gelation and particulate formation, especially under cGMP conditions so that it may be applied for human use.

### SUMMARY OF THE INVENTION

The present disclosure provides processes for preparing glycated chitosan, comprising:
(a) reacting chitosan with a monosaccharide or a reducing oligosaccharide to form a first mixture; and
(b) adding the reducing agent to the first mixture from step (a) to form a second mixture, wherein the reducing agent is added in a subsurface manner.

The present disclosure also provides aqueous compositions comprising at least about 1% w/w glycated chitosan, less than 1.0 mg/mL saccharide, and less than 300 ppm residual boron. The present disclosure also provides aqueous compositions comprising at least about 1% w/w glycated chitosan, less than 1.0 mg/mL saccharide, and less than 20 ppm residual boron. The aqueous composition may be obtained by the processes disclosed herein.

### DETAILED DESCRIPTION OF THE INVENTION

The present disclosure provides, in various aspects, methods for the scaled-up synthesis of glycated chitosans that surprisingly allow for consistent production of desired products, particularly under cGMP.

Chitosan is synthesized from chitin, a linear homopolymer composed of N-acetylglucosamine units joined by β-1-4 glycosidic bonds, by removing acetyl groups from chitin using various processes which include, but are not limited to, exposure to aqueous sodium hydroxide or potassium hydroxide. As known in the art, the deacetylation reaction does not always proceed to completion, and a portion of the originally-present acetyl groups may remain on the chitosan glucosamine units after the reaction. The presently disclosed methods for preparing glycated chitosan may use chitosan of any suitable degree of deacetylation, and the resulting glycated chitosans will retain an amount of acetyl groups from the chitin starting material.

Glycated chitosan, as indicated above, refers to the products resulting from the reaction between the free amino groups of chitosan and the carbonyl groups of reducing monosaccharides and/or oligosaccharides. The products of this reaction (mainly a mixture of Schiff bases, i.e. the carbon atom of the carbonyl group double-bonded to the nitrogen atom of the amino group, and Amadori products, i.e. the carbon atom of said carbonyl group bonded to the nitrogen atom of said amino group by a single bond while an adjacent carbon atom is double-bonded to an oxygen atom) may be used as such or after stabilization by reduction with hydrides, such as sodium borohydride, or by exposure to hydrogen in the presence of suitable catalysts. These reactions are further described in U.S. Pat. No. 5,747,475 and US 2018/312611, both of which are fully incorporated by reference herein.

Glycated chitosan comprises monomers randomly selected from:

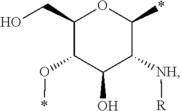

wherein R is selected from:

| R Group | Name of Monomer |
|---|---|
| 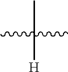 [SMILES: *[H]] | D-glucosamine |
| 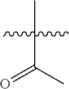 [SMILES: *C(C)=O] | N-acetyl-D-glucosamine |
| 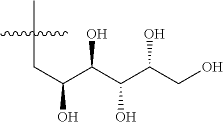 [SMILES: *C[C@H](O)[C@@H](O)[C@@H](O)[C@H](O)CO] | A sugar, such as N-galactosyl-D- glucosamine |

The processes described herein also allow certain features of the product, such as degree of glycation, to be controlled in ways that were not previously possible. Embodiments provided below describe the interrelated effect of conditions such as selection of reducing agent, pH, temperature, concentration, and stirring. Specifically, with respect to the reducing agent, the methods described herein were developed to address the selection, stoichiometry, solution concentration, solution addition rate, and physical method of solution addition. While many reducing agents are known to be effective in reductive amination reactions, the solubility and metal ligating characteristics of chitosan and glycated chitosan as well as the pharmaceutical end-use of glycated chitosan limit the reducing agents that may be used.

The optimal pH for the reduction reaction was surprisingly found to be moderately acidic (e.g., pH less than 5.5). While higher pH values generally avoid the aqueous decomposition of the reducing agent prior to reaction with the imine, glycated chitosan solutions exhibit non-Newtonian behaviors (e.g., gelling) at pH values of 5.5-6 and above. Moreover, while conventional methods adjust the pH only before commencing the reaction, in certain embodiments of the present disclosure, the pH of the reaction is continuously monitored and adjusted, e.g., with acetic acid. This provides both the ideal balance of reducing agent reactivity and decomposition and also a suitable non-gelling environment for the reaction to occur. Maintaining the reaction at a lower pH can shorten the half-life of certain reducing agents (e.g., NaBH₄). However, this is counterbalanced by the advantages of avoiding gelation. This ideal balance of pH also allows for the careful control of percent substitution, which was previously not possible. Reactions to produce GC that are maintained in this ideal pH range can be monitored for percent substitution, and subsequently the dosing of the reducing agent can be adjusted to provide products with the desired substitution rate.

The manner in which the reducing agent solution is dosed to the reaction also influences the outcome of the reaction. Conventional, dropwise addition of a reducing agent solution to the glycated chitosan reaction mixture reduces or eliminates product formation and causes solids to form in the reaction mixture. Without being limited by theory, this is likely due to the inability of titrant solution to effectively break the surface tension of the viscous reaction mixture, creating concentrated microenvironments for reducing agent decomposition and the formation of solids. Adding the reducing agent in a subsurface manner eliminates this problem.

Thus, in certain aspects, the present disclosure provides processes for preparing glycated chitosan, comprising:
(a) reacting chitosan with a monosaccharide or reducing oligosaccharide to form a first mixture; and
(b) adding a reducing agent to the first mixture from step (a) to form a second mixture,
   wherein the reducing agent is added in a subsurface manner.

In certain embodiments, the monosaccharide is selected from trioses, tetroses, pentoses, hexoses, and heptoses. In certain embodiments, the monosaccharide is selected from glucose, glucuronic acid, galactose, fructose, mannose, allose, altrose, idose, talose, fucose, arabinose, gulose, hammelose, lyxose, ribose, rhamnose, threose, xylose, psicose, sorbose, tagatose, glyceraldehyde, dihydroxyacetone, erythrose, threose, erythrulose, mannoheptulose, and sedoheptulose. In certain embodiments, the oligosaccharide is selected from fructo-oligosaccharides (FOS), the galacto-oligosaccharides, (GOS), and mannan-oligosaccharides (MOS). In certain embodiments, the monosaccharide is selected from glucose, galactose, mannose, allose, altrose, idose, talose, fucose, arabinose, gulose, hammelose, lyxose, ribose, rhamnose, threose, xylose, glyceraldehyde, erythrose, and threose. In certain embodiments, the monosaccharide or reducing oligosaccharide is a monosaccharide selected from glucose, ribose, and galactose.

In certain embodiments, the monosaccharide is galactose.

In certain embodiments, the monosaccharide or reducing oligosaccharide is a reducing oligosaccharide comprising 2 monosaccharide units. In certain embodiments, the monosaccharide or reducing oligosaccharide is a reducing oligosaccharide comprising 5 monosaccharide units. In certain embodiments, the monosaccharide or reducing oligosaccharide is a reducing oligosaccharide comprising 2 to 10 monosaccharide units.

In certain embodiments, the reducing oligosaccharide comprises 2 monosaccharide units independently selected from glucose, ribose, and galactose. In certain embodiments, the reducing oligosaccharide comprises 5 monosaccharide units independently selected from glucose, ribose, and galactose. In certain embodiments, the reducing oligosaccharide comprises at least 2 monosaccharide units independently selected from glucose, ribose, and galactose. In certain embodiments, the reducing oligosaccharide comprises 2 to 10 monosaccharide units independently selected from glucose, ribose, and galactose. In certain embodiments, the reducing oligosaccharide consists essentially of 2 to 10 monosaccharide units independently selected from glucose, ribose, and galactose.

In some embodiments, the chitosan has a degree of deacetylation of about 70-95%. In certain embodiments, the chitosan has a degree of deacetylation of about 80%.

In certain embodiments, step (a) comprises contacting the chitosan with a solvent, such as water, to form a solution.

In certain embodiments, step (a) further comprises adjusting the pH of the solution to less than 5.0 before contacting the chitosan with the monosaccharide or the reducing oligosaccharide. In certain embodiments, step (a) further comprises adjusting the pH of the solution to less than 3.0 before contacting the chitosan with the monosaccharide or the reducing oligosaccharide. In certain embodiments, step (a) further comprises adjusting the pH of the solution to less than 1.0 before contacting the chitosan with the monosaccharide or the reducing oligosaccharide.

In certain embodiments, the pH is adjusted by addition of an acid. In certain preferred embodiments, the acid is acetic acid.

In certain embodiments, step (a) is performed for a period of at least about 3 hours. In certain embodiments, step (a) is performed for a period of at least about 6 hours. In certain embodiments, step (a) is performed for a period of at least 9 hours. In certain embodiments, step (a) is performed for a period of at least about 12 hours. In certain embodiments, step (a) is performed for a period of at least about 18 hours. In certain embodiments, step (a) is performed for a period of at least 24 hours. In certain embodiments, step (a) is performed for a period of about 1 hour to about 48 hours. In certain embodiments, step (a) is performed for a period of about 6 hours to about 24 hours. In certain embodiments, step (a) is performed for a period of about 12 hours.

In certain particularly preferred embodiments, step (a) comprises contacting the chitosan with the monosaccharide or the reducing oligosaccharide in a solvent, the solvent comprises water and step (a) is performed for a period of about 12 hours.

In certain embodiments, the reducing agent is a borane or a borohydride. In certain embodiments, the reducing agent is a borohydride. In certain preferred embodiments, the reducing agent is sodium borohydride.

In certain embodiments, the reducing agent is added via a plurality of subsurface addition ports. In certain embodiments, the reducing agent is added via 2 to 6 subsurface addition ports. In certain embodiments, the reducing agent is added via 4 subsurface addition ports.

In certain embodiments, the reducing agent is added over a period of about 4 hours to about 10 hours. In certain embodiments, the reducing agent is added over a period of about 5 hours to about 8 hours. In certain embodiments, the reducing agent is added over a period of about 6 hours to about 7 hours.

In certain preferred embodiments, the reducing agent is added using a dosing pump.

In certain embodiments, step (b) is performed at a temperature of less than 0° C. In certain embodiments, step (b) is performed at a temperature of about 0° C. to about 15° C. In certain embodiments, step (b) is performed at a temperature of about 0° C. to about 10° C. In certain embodiments, step (b) is performed at a temperature of about 3° C. to about 10° C. In certain preferred embodiments, step (b) is performed at a temperature of about 6° C. to about 8° C.

In certain embodiments, step (b) is performed at a pH of about 0.5 to about 5.5. In certain embodiments, step (b) is performed at a pH of about 2.5 to about 5.5. In certain embodiments, step (b) is performed at a pH of about 4.0 to about 4.5. In certain embodiments, step (b) is performed at a pH of at most about 6.0. In certain embodiments, step (b) is performed at a pH of at most about 5.5. In certain embodiments, step (b) is performed at a pH of at most about 5.0. In certain embodiments, step (b) is performed at a pH of at least 1.0. In certain embodiments, step (b) is performed at a pH of at least about 2.0. In certain embodiments, step (b) is performed at a pH of at least about 3.0.

In certain embodiments, step (b) further comprises adjusting the pH of the first mixture to a reduction pH of about 0.5 to about 5.5. In certain embodiments, step (b) further comprises adjusting the pH of the first mixture to a reduction pH of about 2.5 to about 5.5. In certain embodiments, step (b) further comprises adjusting the pH of the first mixture to a reduction pH of about 4.0 to about 4.5. In certain embodiments, step (b) further comprises adjusting the pH of the first mixture to a reduction pH of 6.0. In certain embodiments, step (b) further comprises adjusting the pH of the first mixture to a reduction pH of about 5.5. In certain embodiments, step (b) further comprises adjusting the pH of the first mixture to a reduction pH of about 5.5. In certain embodiments, step (b) further comprises adjusting the pH of the first mixture to a reduction pH of at least 1.0. In certain embodiments, step (b) further comprises adjusting the pH of the first mixture to a reduction pH of at least 2.0. In certain embodiments, step (b) further comprises adjusting the pH of the first mixture to a reduction pH of at least 3.0. In certain embodiments, step (b) is performed at a chitosan concentration of less than 1.0 g/L. In certain embodiments, step (b) is performed at a chitosan concentration of 1.0 g/L to about 20.0 g/L. In certain embodiments, step (b) is performed at a chitosan concentration of 1.0 g/L to about 10.0 g/L. In certain embodiments, step (b) is performed at a chitosan concentration of 5.0 g/L to about 10.0 g/L. In certain embodiments, step (b) is performed at a chitosan concentration of 4.0 g/L to about 6.0 g/L. In certain embodiments, step (b) is performed at a chitosan concentration of 6.0 g/L to about 7.0 g/L. In certain embodiments, step (b) is performed at a chitosan concentration of 7.0 g/L to about 8.0 g/L. In certain embodiments, step (b) is performed at a chitosan concentration of 8.0 g/L to about 9.0 g/L. In certain embodiments, step (b) is performed at a chitosan concentration of 9.0 g/L to about 10.0 g/L.

In certain particularly preferred embodiments, the reducing agent is a borohydride, the reducing agent is added via a plurality of addition ports, and step (b) is performed at a pH of about 4.0 to about 4.5.

In certain embodiments, step (b) further comprises monitoring the pH of the second mixture.

In certain embodiments, step (b) further comprises adjusting the pH of the second mixture to remain at a pH between about 0.5 to about 5.5. In certain embodiments, step (b) further comprises adjusting the pH of the second mixture to remain at a pH between about 2.5 to about 5.5. In certain embodiments, step (b) further comprises adjusting the pH of the second mixture to remain at a pH between about 4.0 to about 4.5.

In certain embodiment, the pH is adjusted by addition of an acid. In certain preferred embodiments, the acid is acetic acid.

In certain embodiments, overhead stirring is used.

In certain embodiments, the second mixture from step (b) is purified by diafiltration. In certain embodiments, the second mixture from step (b) is purified by diafiltration using a 30 kDa cutoff hollow fiber filter.

In certain embodiments, the second mixture from step (b) is purified by ultrafiltration.

In certain embodiments, the molar ratio of the chitosan to the monosaccharide or the reducing oligosaccharide is greater than 1:20. In certain embodiments, the molar ratio of the chitosan to the monosaccharide or the reducing oligosaccharide is about 1:1 to about 1:20. In certain embodiments, the molar ratio of the chitosan to the monosaccharide or the reducing oligosaccharide is about 1:1 to about 1:15. In certain embodiments, the molar ratio of the chitosan to the monosaccharide or the reducing oligosaccharide is about 1:1 to about 1:10. In certain embodiments, the molar ratio of the chitosan to the monosaccharide or the reducing oligosaccharide is about 1:3 to about 1:10. In certain embodiments, the molar ratio of the chitosan to the monosaccharide or the reducing oligosaccharide is about 1:5.

In certain embodiments, the molar ratio of the chitosan to the sodium borohydride is greater than 1:20. In certain embodiments, the molar ratio of the chitosan to the sodium borohydride is about 1:1 to about 1:20. In certain embodiments, the molar ratio of the chitosan to the sodium borohydride is about 1:1 to about 1:15. In certain embodiments, the molar ratio of the chitosan to the sodium borohydride is about 1:1 to about 1:10. In certain embodiments, the molar ratio of the chitosan to the sodium borohydride is about 1:5 to about 1:10. In certain embodiments, the molar ratio of the chitosan to the sodium borohydride is about 1:7 to about 1:9. In certain embodiments, the molar ratio of the chitosan to the sodium borohydride is about 1:8. In certain embodiments, step (a) is performed on a chitosan scale of greater than 10 grams. In certain embodiments, step (a) is performed on a chitosan scale of greater than 1 kilogram. In certain embodiments, step (a) is performed on a chitosan scale of greater than 10 kilograms. In certain embodiments, step (a) is performed on a chitosan scale of about 100 milligrams to about 10 kilograms. In certain embodiments, the step (a) is performed on a chitosan scale of about 1.0 gram to about 1.0 kilograms. In certain embodiments, the step (a) is performed on a chitosan scale of about 20.0 grams to about 100 grams.

In certain aspects, the present disclosure provides aqueous compositions comprising at least about 1% w/w glycated chitosan, less than 1.0 mg/mL saccharide, and less than 300 ppm residual boron. In some embodiments, the aqueous compositions comprise less than 150 ppm residual boron. In some embodiments, the aqueous compositions comprise less than 50 ppm residual boron. In certain aspects, the present disclosure provides aqueous compositions comprising at least about 1% w/w glycated chitosan, less than 1.0 mg/mL saccharide, and less than 20 ppm residual boron. The aqueous composition may be obtained by the processes disclosed herein.

In some embodiments, the glycated chitosan has a degree of deacetylation of about 70-95%. In certain embodiments, the glycated chitosan has a degree of deacetylation of about 80%.

In certain embodiments, the residual monosaccharide or reducing oligosaccharide is present at a concentration of less than 0.85 mg/mL, less than 0.7 mg/mL, less than 0.65 mg/mL, or less than 0.5 mg/mL. In certain embodiments, the residual monosaccharide or reducing oligosaccharide is about 0.65 mg/mL.

In certain embodiments, the glycated chitosan has a molecular weight of at least 50 kDa. In certain embodiments, the glycated chitosan has a molecular weight of at least 100 kDa. In certain embodiments, the glycated chitosan has a molecular weight of at least 200 kDa. In certain embodiments, the glycated chitosan has a molecular weight of at least 300 kDa. In certain embodiments, the glycated chitosan has a molecular weight of at least 500 kDa. In certain embodiments, the glycated chitosan has a molecular weight of about 10 kDa to about 1000 kDa. In certain embodiments, the glycated chitosan has a molecular weight of about 50 kDa to about 350 kDa. In certain embodiments, the glycated chitosan has a molecular weight of about 80 kDa to about 200 kDa. In certain embodiments, the glycated chitosan has a molecular weight of about 100 kDa to about 150 kDa.

In certain embodiments, the degree of glycation, such as with galactose, can be controlled by varying the concentration of the sodium borohydride solution that is added to the imine mixture in the processes described herein. An increased sodium borohydride concentration generally provides a greater degree of glycation. In certain embodiments, the glycated chitosan has a degree of glycation of less than 1%. In certain embodiments, the glycated chitosan has a degree of glycation of at least 1%. In certain embodiments, the glycated chitosan has a degree of glycation of about 2% to about 5%. In certain embodiments, the glycated chitosan has a degree of glycation of about 1% to about 20%. In certain embodiments, the glycated chitosan has a degree of glycation of about 10% to about 20%. In certain embodiments, the glycated chitosan has a degree of glycation of about 1% to about 10%. In certain embodiments, the glycated chitosan has a degree of glycation of greater than 10%. In certain embodiments, the glycated chitosan has a degree of glycation of about 1% to about 5%. In certain embodiments, the glycated chitosan has a degree of glycation of about 5% to about 10%. In certain embodiments, the glycated chitosan has a degree of glycation of about 2% to about 8%. In certain embodiments, the glycated chitosan has a degree of glycation of about 3% to about 7%. In certain embodiments, the glycated chitosan has a degree of glycation of about 3%. In certain embodiments, the glycated chitosan has a degree of glycation of about 4%. In certain embodiments, the glycated chitosan has a degree of glycation of about 5%. In certain embodiments, the glycated chitosan has a degree of glycation of about 6%. In certain embodiments, the glycated chitosan has a degree of glycation of about 7%. In certain embodiments, the glycated chitosan has a degree of glycation of about 8%. In certain embodiments, the glycated chitosan has a degree of glycation of about 9%. In certain embodiments, the glycated chitosan has a degree of glycation of about 10%. In certain embodiments, the glycated chitosan has a degree of glycation of about 15%. In certain embodiments, the glycated chitosan has a degree of glycation of about 20%. In certain embodiments, the glycated chitosan has a degree of glycation of at least about 30%.

In certain embodiments, the glycated chitosan has a composition of about 1% to about 20% N-galactosyl-D-glucosamine; about 1% to about 30% N-acetyl-glucosamine; and about 50% to about 98% D-glucosamine.

In certain embodiments, the glycated chitosan is glycated with galactose. In certain embodiments, the glycated chitosan has a degree of galactation of about 1% to about 5%. In certain embodiments, the glycated chitosan has a degree of galactation of about 3% to about 4%.

In certain embodiments, the residual boron present in the aqueous composition is less than 300 ppm residual boron. In certain embodiments, the residual boron present in the aqueous composition is less than 150 ppm residual boron. In certain embodiments, the residual boron present in the aqueous composition is less than 50 ppm. In certain embodiments, the residual boron is less than 25 ppm. In certain embodiments, the residual boron is less than 15 ppm. In certain embodiments, the residual boron is less than 10 ppm. In certain embodiments, the residual boron is less than 5 ppm. In certain embodiments, the residual boron is less than 1 ppm.

In certain embodiments, the aqueous composition comprises at least about 1.2% w/w glycated chitosan.

In certain embodiments, the bacterial enumeration in the aqueous composition is less than 10 CFU/10 mL. In certain embodiments, the bacterial enumeration is less than 5 CFU/10 mL. In certain embodiments, the bacterial enumeration is less than 1 CFU/10 mL.

In certain embodiments, the endotoxin level in the aqueous composition is less than 7.5 EU/mg. In certain embodiments, the endotoxin level in the aqueous composition is less than 5 EU/mg. In certain embodiments, the endotoxin level in the aqueous composition is less than 1.5 EU/mg. In certain embodiments, the endotoxin level in the aqueous composition is less than 1.0 EU/mg. In certain embodiments, the endotoxin level in the aqueous composition is less than 0.5 EU/mg.

### Definitions

“Borane” is an art-recognized term and refers a class of synthetic hydrides of boron with generic formula BₓH_{y}. Representative boranes are BH₃, B₂H₆, and B₄H₁₀.

“Borohydride” is an art-recognized term and refers to the anion BH₄⁻ and its salts. Borohydride is also the term used for compounds containing BH₄₋ₙX⁻ₙ, for example cyanoborohydride (B(CN)H₃⁻), triacetoxyborohydride, and triethylborohydride (B(C₂H₅)₃H⁻). Representative borohydrides are lithium borohydride, potassium borohydride, zinc borohydride, and sodium borohydride.

“Subsurface addition” as used herein refers to adding a liquid directly to another liquid without contacting the gas phase. This may be achieved, for example, by adding the liquid using a dosing pump via one or more subsurface addition ports.

“Degree of glycation” as used herein refers to the number of glycated amine groups in a glycated chitosan divided by the total number of polymer subunits. When a degree of glycation is specified herein with respect to a polymer ensemble (for example a polymer mixture in a reaction or product mixture), that is a reference to percentage of glycated units in the ensemble with respect to the total number of units. The degree of glycation may be measured, for example, using high-field ¹H NMR spectrometry. Resonances specific to glycated residues as well as the polymer backbone are integrated, with the ratio of the resonances representing the degree of glycation of the sample.

As used herein, a “number average” of a property of a polymer chain refers to the unweighted mean of that property across a polymer ensemble. Thus, for example, the “number average molecule weight” of a polymer ensemble may be expressed by the equation:

$${\overline{M}}_{n} = \frac{{\sum }_{i} {N}_{i} {M}_{i}}{{\sum }_{i} {N}_{i}}$$

where Nᵢ is the number of molecules of molecular weight Mᵢ. Weight average quantities may be determined by any suitable technique. For example, the number average molecular weight may be determined by gel permeation chromatography (also known as size exclusion chromatography) or viscometry. Other number averaged quantities may, for example, be derived from the number average molecular weight. For example, the number average of the number of repeat units in a polymer ensemble (also known as the number average degree of polymerization) may be calculated as the ratio of the number average molecular weight to the molecular weight of the repeat unit (appropriately averaged if necessary). The number average of the degree of polymerization of a polymer ensemble may also be measured directly, for example by end-group analysis.

As used herein, a “weight average” of a property of a polymer chain refers to the mean of that property across a polymer ensemble, weighted by the molecular weight of the polymer chains. Thus, for example, the “weight average molecular weight” of a polymer ensemble may be expressed by the equation:

$${\overline{M}}_{w} = \frac{{\sum }_{i} {N}_{i} {M}_{i}^{2}}{{\sum }_{i} {N}_{i} {M}_{i}}$$

where Nᵢ is the number of molecules of molecular weight Mᵢ. Weight average quantities may be determined by any suitable technique. For example, the weight average molecular weight may be determined by light scattering, small angle neutron scattering, X-ray scattering, or sedimentation velocity. Other weight averaged quantities may, for example, be derived from the weight average molecular weight. For example, the weight average of the number of repeat units in a polymer ensemble (also known as the weight average degree of polymerization) may be calculated as the ratio of the weight average molecular weight to the molecular weight of the repeat unit (appropriately averaged if necessary).

### EXAMPLES

The invention now being generally described, it will be more readily understood by reference to the following examples, which are included merely for purposes of illustration of certain aspects and embodiments of the present invention and are not intended to limit the invention.

### Example 1. Scaled Up Process

An appropriately sized glass-lined vessel, equipped with a cooling jacket and overhead stirring was charged with 8% aqueous acetic acid (2 L). The resulting solution was stirred and charged with chitosan (35.0 g, 206 mmol) followed by D-galactose (188.0 g 1044 mmol). The resulting suspension was charged with additional 8% aqueous acetic acid (1.5 L) and stirred vigorously at ambient temperature for a minimum of 12 hours. The resulting solution was cooled to 6-8° C. using jacketed cooling then diluted with water (2.5 L). The pH of the solution was adjusted to a value of 4 using a 2.0 M aqueous solution of sodium hydroxide (added in 25 mL aliquots until the desired pH was achieved). The resulting solution was then dosed with a 0.87 M solution of sodium borohydride in 4.38 mM aqueous sodium hydroxide over a period of 6.5-7 hours using a calibrated dosing pump. The borohydride solution was added in a subsurface manner, via 4 subsurface addition ports. Gas evolution and foaming was observed throughout the titration and the stirring speed was adjusted to ensure the foaming did not exceed the height of the cooling jacket. The reaction was monitored for pH and temperature during the entire borohydride titration, with the pH being adjusted with glacial acetic acid (in 40 mL aliquots) when the pH reached 4.5 and the cooling adjusted based on the temperature read via a subsurface thermowell. Upon completion of the sodium borohydride titration, the resulting solution was allowed to warm to ambient temperature overnight with reduced stirring. The resulting solution was then purified by diafiltration using a 30 kDa cutoff hollow fiber filter. The solution was diluted to 12 L with water, then ultrafiltered to 6 L, followed by 16 passes of the addition and ultrafiltration of water (2 L, 32 L in total). System pressure was monitored throughout the diafiltration and ultrafiltration steps, reducing system flow if the pre-filter pressure reached 50 psi. The resulting solution was ultrafiltered to a volume of ˜2.8 L, then passed through a 0.22-micron cartridge filter into a sterile biobag to provide the final solution of glycated chitosan with a degree of glycation of 3.5%.

### Example 2. Smaller Scaled Up Batch

An appropriately sized glass-lined vessel, equipped with a cooling jacket and overhead stirring was charged with 8% aqueous acetic acid (130 g). The resulting solution was stirred and charged with chitosan (2.33 g, 13.71 mmol) followed by D-galactose (12.53 g, 69.58 mmol). The resulting suspension was charged with additional 8% aqueous acetic acid (100 g) and stirred vigorously at ambient temperature for a minimum of 12 hours. The resulting solution was cooled to 6-8° C. using jacketed cooling then diluted with water (167 g). The pH of the solution was adjusted to a value of 4 using a 2.0 M aqueous solution of sodium hydroxide (added in 2.4 mL aliquots until the desired pH was achieved). The resulting solution was then dosed with a 0.91 M solution of sodium borohydride in 4.72 mM aqueous sodium hydroxide over a period of 6.5-7 hours using a calibrated dosing pump. The borohydride solution was added in a subsurface manner, via 1 subsurface addition ports. Gas evolution and foaming was observed throughout the titration and the stirring speed was adjusted to ensure the foaming did not exceed the height of the cooling jacket. The reaction was monitored for pH and temperature during the entire borohydride titration, with the pH being adjusted with glacial acetic acid (in 2.7 mL aliquots) when the pH reached 4.5 and the cooling adjusted based on the temperature read via a subsurface thermowell. Upon completion of the sodium borohydride titration, the resulting solution was allowed to warm to ambient temperature overnight with reduced stirring. The resulting solution was then purified by diafiltration using a 30 kDa cutoff hollow fiber filter. The solution was diluted to 0.8 L with water, then ultrafiltered to 0.4 L, followed by 16 passes of the addition and ultrafiltration of water (0.135 L, 2.16 L in total). System pressure was monitored throughout the diafiltration and ultrafiltration steps, reducing system flow if the pre-filter pressure reached 38 psi. The resulting solution was ultrafiltered to a volume of ˜0.4 L, then passed through a 0.22-micron cartridge filter into a sterile biobag to provide the final solution of glycated chitosan with a degree of glycation of 3.7%.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A process for preparing glycated chitosan, comprising
(a) reacting chitosan with a monosaccharide or reducing oligosaccharide to form a first mixture; and
(b) adding a reducing agent to the first mixture from step (a) to form a second mixture,
wherein the reducing agent is added in a subsurface manner.

2. The process of claim 1, wherein the monosaccharide or reducing oligosaccharide is a monosaccharide selected from glucose, ribose, and galactose.

3. The process of claim 2, wherein the monosaccharide is galactose.

4. The process of claim 1, wherein the monosaccharide or reducing oligosaccharide is a reducing oligosaccharide comprising 2 to 10 monosaccharide units.

5. The process of claim 4, wherein the reducing oligosaccharide comprises 2 to 10 monosaccharide units independently selected from glucose, ribose, and galactose.

6. The process of any one of claims 1-5, wherein step (a) comprises contacting the chitosan with a solvent to form a solution.

7. The process of claim 6, wherein the solvent comprises water.

8. The process of claim 6, wherein the solvent is water.

9. The process of claim 7 or 8, comprising adjusting the pH of the solution to less than 5.0 before contacting the chitosan with the monosaccharide or the reducing oligosaccharide.

10. The process of claim 9, comprising adjusting the pH of the solution to less than 3.0 before contacting the chitosan with the monosaccharide or the reducing oligosaccharide.

11. The process of claim 10, comprising adjusting the pH of the solution to less than 1.0 before contacting the chitosan with the monosaccharide or the reducing oligosaccharide.

12. The process of any one of claims 9-11, wherein the pH is adjusted by addition of an acid.

13. The process of claim 12, wherein the acid is acetic acid.

14. The process of any one of claims 1-13, wherein step (a) is performed for a period of about 1 hour to about 48 hours.

15. The process of claim 14, wherein step (a) is performed for a period of about 6 hours to about 24 hours.

16. The process of claim 15, wherein step (a) is performed for a period of about 12 hours.

17. The process of claim 1, wherein step (a) comprises contacting the chitosan with the monosaccharide or the reducing oligosaccharide in a solvent, the solvent comprises water and step (a) is performed for a period of about 12 hours.

18. The process of any one of claims 1-17, wherein the reducing agent is a borane or a borohydride.

19. The process of claim 18, wherein the reducing agent is a borohydride.

20. The process of claim 19, wherein the reducing agent is sodium borohydride.

21. The process of any one of claims 1-20, wherein the reducing agent is added via a plurality of subsurface addition ports.

22. The process of claim 21, wherein the reducing agent is added via 2 to 6 subsurface addition ports.

23. The process of claim 22, wherein the reducing agent is added via 4 subsurface addition ports.

24. The process of any one of claims 1-23, wherein the reducing agent is added over a period of about 4 hours to about 10 hours.

25. The process of claim 24, wherein the reducing agent is added over a period of about 5 hours to about 8 hours.

26. The process of claim 25, wherein the reducing agent is added over a period of about 6 hours to about 7 hours.

27. The process of any one of claims 1-26, wherein the reducing agent is added using a dosing pump.

28. The process of any one of claims 1-27, wherein step (b) is performed at a temperature of about 0° C. to about 15° C.

29. The process of claim 28, wherein step (b) is performed at a temperature of about 3° C. to about 10° C.

30. The process of claim 29, wherein step (b) is performed at a temperature of about 6° C. to about 8° C.

31. The process of any one of claims 1-30, wherein step (b) is performed at a pH of about 0.5 to about 5.5.

32. The process of claim 31, wherein step (b) is performed at a pH of about 2.5 to about 5.5.

33. The process of claim 32, wherein step (b) is performed at a pH of about 4.0 to about 4.5.

34. The process of any one of claims 1-33, wherein step (b) is performed at a chitosan concentration of 1.0 g/L to about 20.0 g/L.

35. The process of claim 34, wherein step (b) is performed at a chitosan concentration of 5.0 g/L to about 10.0 g/L.

36. The process of claim 35, wherein step (b) is performed at a chitosan concentration of 7.0 g/L to about 8.0 g/L.

37. The process of any one of claims 1-17, wherein the reducing agent is a borohydride, the reducing agent is added via a plurality of addition ports, and step (b) is performed at a pH of about 4.0 to about 4.5.

38. The process of any one of claims 1-37, wherein step (b) further comprises monitoring the pH of the second mixture.

39. The process of claim 1-38, wherein step (b) further comprises adjusting the pH of the second mixture to remain at a pH between about 0.5 to about 5.5.

40. The process of claim 39, wherein step (b) further comprises adjusting the pH of the second mixture to remain at a pH between about 2.5 to about 5.5.

41. The process of claim 40, wherein step (b) further comprises adjusting the pH of the second mixture to remain at a pH between about 4.0 to about 4.5.

42. The process of any one of claims 38-40, wherein the pH is adjusted by addition of an acid.

43. The process of claim 42, wherein the acid is acetic acid.

44. The process of any one of claims 1-43, wherein overhead stirring is used.

45. The process of any one of claims 1-44, wherein the second mixture from step (b) is purified by diafiltration.

46. The process of claim 45, wherein the second mixture from step (b) is purified by diafiltration using a 30 kDa cutoff hollow fiber filter.

47. The process of any one of claims 1-44, wherein the second mixture from step (b) is purified by ultrafiltration.

48. The process of any one of claims 1-47, wherein the molar ratio of the chitosan to the monosaccharide or the reducing oligosaccharide is about 1:1 to about 1:20.

49. The process of claim 48, wherein the molar ratio of the chitosan to the monosaccharide or the reducing oligosaccharide is about 1:3 to about 1:10.

50. The process of claim 49, wherein the molar ratio of the chitosan to the monosaccharide or the reducing oligosaccharide is about 1:5.

51. The process of any one of claims 1-50, wherein the molar ratio of the chitosan to the sodium borohydride is about 1:1 to about 1:20.

52. The process of claim 51, wherein the molar ratio of the chitosan to the sodium borohydride is about 1:5 to about 1:10.

53. The process of claim 52, wherein the molar ratio of the chitosan to the sodium borohydride is about 1:7 to about 1:9.

54. The process of any one of claims 1-53, wherein step (a) is performed on a chitosan scale of about 100 milligrams to about 10 kilograms.

55. The process of claim 54, wherein the step (a) is performed on a chitosan scale of about 1.0 gram to about 1.0 kilograms.

56. The process of claim 55, wherein the step (a) is performed on a chitosan scale of about 20.0 grams to about 100 grams.

57. The process of any one of claims 1-56, wherein the chitosan has a degree of deacetylation of about 70-95%.

58. The process of any one of claims 1-57, wherein the chitosan has a degree of deacetylation of about 80%.

59. An aqueous composition comprising at least about 1% w/w glycated chitosan, less than 1.0 mg/mL saccharide, and less than 20 ppm residual boron.

60. An aqueous composition comprising at least about 1% w/w glycated chitosan, less than 1.0 mg/mL saccharide, and less than 50 ppm residual boron.

61. An aqueous composition comprising at least about 1% w/w glycated chitosan, less than 1.0 mg/mL saccharide, and less than 150 ppm residual boron.

62. An aqueous composition comprising at least about 1% w/w glycated chitosan, less than 1.0 mg/mL saccharide, and less than 300 ppm residual boron.

63. An aqueous composition comprising at least about 1% w/w glycated chitosan, less than 1.0 mg/mL residual monosaccharide or reducing oligosaccharide, and less than 20 ppm residual boron, wherein the aqueous composition is obtainable by the process of any one of claims 1-56.

64. An aqueous composition comprising at least about 1% w/w glycated chitosan, less than 1.0 mg/mL residual monosaccharide or reducing oligosaccharide, and less than 50 ppm residual boron, wherein the aqueous composition is obtainable by the process of any one of claims 1-56.

65. An aqueous composition comprising at least about 1% w/w glycated chitosan, less than 1.0 mg/mL residual monosaccharide or reducing oligosaccharide, and less than 150 ppm residual boron, wherein the aqueous composition is obtainable by the process of any one of claims 1-56.

66. An aqueous composition comprising at least about 1% w/w glycated chitosan, less than 1.0 mg/mL residual monosaccharide or reducing oligosaccharide, and less than 300 ppm residual boron, wherein the aqueous composition is obtainable by the process of any one of claims 1-56.

67. The aqueous composition of any one of claims 59-63, wherein the residual monosaccharide or reducing oligosaccharide is present at a concentration of less than 0.85 mg/mL.

68. The aqueous composition of claim 67, wherein the concentration of the residual monosaccharide or reducing oligosaccharide is present at a concentration of less than 0.7 mg/mL.

69. The aqueous composition of claim 68, wherein the concentration of the residual monosaccharide or reducing oligosaccharide is present at a concentration of about 0.65 mg/mL.

70. The aqueous composition of claim 69, wherein the glycated chitosan has a molecular weight of about 10 kDa to about 1000 kDa.

71. The aqueous composition of any one of claims 59-70, wherein the glycated chitosan has a molecular weight of about 50 kDa to about 350 kDa.

72. The aqueous composition of claim 71, wherein the glycated chitosan has a molecular weight of about 80 kDa to about 200 kDa.

73. The aqueous composition of claim 72, wherein the glycated chitosan has a molecular weight of about 100 kDa to about 150 kDa.

74. The aqueous composition of any one of claims 59-73, wherein the glycated chitosan has a degree of glycation of at least about 30%.

75. The aqueous composition of any one of claims 59-73, wherein the glycated chitosan has a degree of glycation of about 1% to about 10%.

76. The aqueous composition of claim 75, wherein the glycated chitosan has a degree of glycation of about 3% to about 7%.

77. The aqueous composition of claim 76, wherein the glycated chitosan has a degree of glycation of about 5%.

78. The aqueous composition of any one of claims 59-77, wherein the glycated chitosan is glycated with galactose.

79. The aqueous composition of any one of claims 59-78, wherein the residual boron present in the aqueous composition is less than 15 ppm.

80. The aqueous composition of any one of claims 59-78, wherein the residual boron present in the aqueous composition is less than 50 ppm.

81. The aqueous composition of any one of claims 59-78, wherein the residual boron present in the aqueous composition is less than 150 ppm.

82. The aqueous composition of any one of claims 59-78, wherein the residual boron present in the aqueous composition is less than 300 ppm.

83. The aqueous composition of any one of claims 59-82, wherein the aqueous composition comprises at least about 1.2% w/w glycated chitosan.

84. The aqueous composition of any one of claims 59-83, wherein the bacterial enumeration in the aqueous composition is less than 10 CFU/10 mL.

85. The aqueous composition of claim 84, wherein the bacterial enumeration in the aqueous composition is less than 5 CFU/10 mL.

86. The aqueous composition of claim 85, wherein the bacterial enumeration in the aqueous composition is less than 1 CFU/10 mL.

87. The aqueous composition any one of claims 59-86, wherein the endotoxin level in the aqueous composition is less than 7.5 EU/mg.

88. The aqueous composition any one of claims 59-86, wherein the endotoxin level in the aqueous composition is less than 5 EU/mg.

89. The aqueous composition any one of claims 59-87, wherein the endotoxin level in the aqueous composition is less than 1.5 EU/mg.

90. The aqueous composition of claim 89, wherein the endotoxin level in the aqueous composition is less than 1.0 EU/mg.

91. The aqueous composition of claim 90, wherein the endotoxin level in the aqueous composition is less than 0.5 EU/mg.

92. The aqueous composition of any one of claims 59-91, wherein the glycated chitosan has a degree of deacetylation of about 70-95%.

93. The aqueous composition of claim 92, wherein the glycated chitosan has a degree of deacetylation of about 80%.
